(19) United States
(12) Patent Application Publication
(10) Pub. No.: US 2026/0272506 A1
(43) Pub. Date: Sep. 17, 2026

(54) BODY CAVITY, DUCT, OR VESSEL ENTRY INDICATION DEVICE

(71) Applicant: Cannulight Technology Limited, Auckland (NZ)

(72) Inventors: James Robert STONE, Auckland (NZ); Ian Douglas Makinson, Auckland (NZ); Jeffrey John Sharp, Auckland (NZ); Bruce MORRIS, Auckland (NZ); Michael Anthony MUNLEY, Auckland (NZ); Keith Colson, Auckland (NZ)

(73) Assignee: Cannulight Technology Limited, Auckland (NZ)

(21) Appl. No.: 19/166,762

(22) PCT Filed: Mar. 21, 2024
(86) PCT No.: PCT/NZ2024/050031

§ 371 Date: Sep. 18, 2025

(30) Foreign Application Priority Data
Mar. 21, 2023 (GB) 2304100.7
Dec. 4, 2023 (AU) 2023903914

(51) Int. Cl. A61B 17/34; A61M 25/00; A61M 25/06

(52) U.S. Cl. CPC A61B 17/3403; A61B 17/3421; A61M 25/0606; A61M 2025/0001

(57) ABSTRACT

A device to indicate entry of a needle into a body cavity, duct, or vessel of a human or non-human animal has a pressure sensor configured to be in fluid communication with a needle bore of the needle in use, and a selectively activated indicator. The device is configured to activate the indicator while pressure acting on the pressure sensor is at or above a minimum pressure threshold, wherein the minimum pressure threshold is about 25 cmH₂O.

### TECHNICAL FIELD

The present technology relates to a device for indicating when an invasive device such as a catheter needle enters a body cavity, duct, or vessel of a human or non-human animal vessel—more particularly a blood vessel (e.g., a vein or artery).

### BACKGROUND

Peripheral intravenous catheters are the most commonly used invasive devices for the purpose of obtaining blood samples, or to administer drugs, fluids and blood products. Both intravenous cannulation and arterial cannulation involve puncturing a vein or an artery with an intravascular needle, then sliding a plastic tube (catheter) over the needle and into the said vein or artery. The catheter then remains inside the vein or artery, whilst the needle is discarded.

It is not uncommon for several punctures to be required in order to achieve successful catheterization. If the tip of the needle leaves the inside of the vein or artery during cannulation, for example due to patient movement or a double puncture of a vein, before the catheter is advanced, it is then impossible to successfully advance the catheter into the vein.

These successive attempts can produce pain and delay the start of diagnostic treatments or tests. Additionally, repeated punctures can degrade vascular walls which complicate subsequent attempts. This may produce serious complications associated with vascular access, such as bruising, catheter-associated infection, extravasation, haemorrhages, phlebitis, and sepsis.

Devices for indicating the position of a needle have been proposed. However, such devices are not considered sufficiently reliable so as to be clinically useful, and/or are cost prohibitive due to complexity of components and construction, and/or are poorly suited to use in high pressure scenarios such as when providing emergency medical treatment at a point of wounding in a combat or training environment.

It is an object of the present disclosure to address at least one of the foregoing problems or at least to provide the public with a useful choice.

Further aspects and advantages of the present disclosure will become apparent from the ensuing description which is given by way of example only.

### SUMMARY

According to an aspect of the present technology there is provided a device to indicate entry of a needle into a body cavity, duct, or vessel of a human or non-human animal, the device comprising:
a pressure sensor configured to be in fluid communication with a needle bore of the needle in use; and
at least one selectively activated indicator,
wherein the device is configured to activate the indicator while pressure acting on the pressure sensor is above a minimum threshold.

In examples the device may be configured to deactivate the indicator while the pressure acting on the pressure sensor is below minimum threshold.

Examples of the device of the present technology may be configured for use in various body cavities, ducts, or vessels of a human or non-human animal. While examples of the present technology may be configured for use in indicating entry of a needle into a blood vessel of a human (e.g., a vein or artery), it should be appreciated that principles of those examples may be adapted for use in other body cavities, ducts, or vessels.

According to another aspect of the present technology there is provided a device to indicate entry of a needle into a body cavity, duct, or vessel of a human or non-human animal, the device comprising:
a pressure sensor configured to be in fluid communication with a needle bore of the needle in use; and
at least one selectively activated indicator,
wherein the device is configured to activate the indicator while pressure acting on the pressure sensor is about 25 cmH₂O and above.

In examples, the device may be configured to activate the at least one indicator while pressure acting on the pressure sensor is about 30 cmH₂O and above.

In examples, the device may be configured to activate the at least one indicator at all pressures from the minimum threshold through to least 100 cmH₂O. In examples, the device may be configured to activate the indicator at all pressures from the minimum pressure through to at least about 250 cmH₂O. In examples, the device may be configured to activate the at least one indicator at all pressures from the minimum threshold through to about 500 cmH₂O.

In examples, the at least one indicator may be configured to output a visual signal when activated. For example, the at least one indicator may be configured to output light when activated. In examples, the indicator may comprise a light configured to output light in the visible spectrum. In examples, the indicator may comprise a light configured to output light in the non-visible spectrum. In examples the indicator may output near-infrared or infrared light when activated.

In examples, the at least one indicator may be configured to output an audible signal when activated.

In examples, the pressure sensor may comprise at least one diaphragm that is deformable under fluid pressure from a first configuration to a second configuration, wherein in the first configuration the indicator is not activated, and in the second configuration the indicator is activated. In examples, in the second configuration the diaphragm closes a switch to activate the indicator.

In examples, the device may comprise at least one flexible printed circuit. In examples, the device may be configured such that in its second configuration the diaphragm displaces at least a movable portion of the flexible printed circuit to complete an electrical circuit and activate the indicator.

In examples, the pressure sensor may comprise a digital pressure sensor. In examples, the digital pressure sensor may be configured as a pressure switch, outputting a signal to activate the indicator when the sensed pressure is above the minimum threshold. In examples the pressure sensor may output a signal indicative of pressure to a controller, and the controller may selectively activate the indicator based on a determination of whether the pressure is above the minim threshold.

In examples the device may comprise a body containing the pressure sensor. In examples comprising at least one diaphragm, the body may comprise a first internal cavity between a first face of the diaphragm and a fluid port in fluid communication with the needle bore of the needle in use. In examples the body may comprise a second internal cavity formed in part by a second face of the diaphragm opposing the first face. In examples the body may comprise at least one air port between the second cavity and an exterior of the body. In examples the at least one air port may be provided in an end surface of the body distal from the fluid port. In examples the body comprises a plurality of air ports.

In examples the fluid port may comprise an inlet at an exterior of the body and an outlet at the first internal cavity. In examples the inlet and the outlet of the fluid port may be axially aligned with a central portion of the diaphragm.

In examples, the device may comprise at least one battery. In examples the at least one battery may be an alkaline battery. In examples the at least one battery may be a button cell. In examples the device may include a plurality of batteries. In examples the plurality of batteries may be stacked in series. In examples the plurality of batteries may be provided along a longitudinal axis between a first end of the body and a second end of the body.

According to another aspect of the present technology there is provided a device to indicate entry of a needle into a body cavity, duct, or vessel of a human or non-human animal, the device comprising:
a pressure sensor configured to be in fluid communication with a needle bore of the needle in use; and
at least one selectively activated indicator, wherein the at least one selectively activated indicator has at least a first indicator output and a second indicator output;
wherein the device is configured to:
   activate the first indicator output when the pressure acting on the pressure sensor is within a first pressure range; and
   activate the second indicator output when the pressure acting on the pressure sensor is within a second pressure range, wherein the second pressure range is greater than the first pressure range.

In examples, the first pressure range may be indicative of central venous pressure (CVP). In examples the second pressure range may be indicative of non-CVP. In examples, the first pressure range may be from about 3 cmH₂O to about 40 cmH₂O. in examples the second pressure range may be from about 40 cmH₂O and above.

In examples the device comprises a first selectively activated indicator providing the first indicator output and a second selectively activated indicator providing the second indicator output.

In examples, the pressure sensor may comprise at least two diaphragms, each diaphragm configured to be deformable under fluid pressure from a first configuration to a second configuration. In examples, the device may be configured such that the first indicator output is activated when a first diaphragm is in the second configuration, and a second diaphragm is in the first configuration. In examples, the device may be configured such that the second indicator output is activated when the first diaphragm is in the second configuration, and the second diaphragm is in the second configuration. In examples the at least two diaphragms may be arranged in stages.

In examples the device may comprise at least one flexible printed circuit portion, comprising a first contact portion configured to contact the first diaphragm when the first diaphragm is in the second configuration, and a second contact portion configured to contact the second diaphragm when the second diaphragm is in the second configuration.

In examples, the pressure sensor may comprise a digital pressure sensor. In examples, the digital pressure sensor may be configured as a pressure switch, outputting a first signal to activate the first indicator output when the sensed pressure is the within the first pressure range, and outputting a second signal to activate the second indicator output when the sensed pressure is the within the second pressure range. In examples the pressure sensor may output a signal indicative of pressure to a controller, and the controller may selectively activate the first indicator output or the second indicator output at least one indicator based on a determination of whether the pressure is within the first pressure range or the second pressure range.

In examples the at least one selectively activated indicator may be a light. In examples the first indicator output may be a first colour and the second indicator output may be a second colour different to the first colour. In examples the first colour may be green, and the second colour may be red.

According to another aspect of the present technology there is provided a device to indicate entry of a needle into a human or animal vessel or cavity comprising:
a diaphragm that is deformable under fluid pressure from a first configuration to a second configuration;
an electrical circuit having at least one powerable indicator; wherein
when the diaphragm is in its first configuration the indicator is not powered;
when the diaphragm is in its second configuration the indicator is powered, and
wherein deformation of the diaphragm into its second configuration completes the electrical circuit and powers the indicator.

By providing a diaphragm that touches a surface so as to complete an electrical circuit a simply constructed and inexpensive indicating device may be created. Further, by providing a powered indicator that is more readily detectable in comparison to prior art indicators a user is able to concentrate on the task at hand (for example cannulation) rather than concentrating on monitoring whether or not an indicator is activated. Beneficially a powered indicator may operate in low light situations such as battlefields or other emergency non-hospital environments. A visual indicator such as a light (such as an LED) may be observed by a user's peripheral vision. An audio indicator such as a buzzer may be heard by a user regardless of where they are looking.

In examples the electrical circuit may comprise at least one flexible printed circuit portion, and in its second configuration the diaphragm displaces a movable part of the flexible printed circuit such that the movable part touches a surface so as to complete the electrical circuit and power the indicator.

In examples the surface may be another part of the flexible printed circuit, or a terminal of a battery.

In examples a spacer may be provided to determine a distance of travel for the movable part of the flexible printed circuit.

In examples the diaphragm may comprise a conductive material that connects the electrical circuit when the diaphragm is in its second position. In examples the diaphragm may be substantially formed of conductive elastomeric material.

In examples when the diaphragm is in its second position it touches a single contact to connect the circuit, or when the diaphragm is in its second position it touches two contacts to connect the circuit. In examples, when the diaphragm is in its second position it touches and activates a pressure-operated switch to connect the circuit.

In examples a diaphragm may be retained over a projection, wherein a face of the projection comprises an outlet for fluid to deform the diaphragm.

In examples a face of the projection may comprise a substantially central outlet for fluid to deform the diaphragm.

In examples a face of the projection comprises at least one indentation. Provision of at least one indentation may reduce the possibility of a membrane being hindered from moving by sticking to the face of the projection. For example, a face of the projection may be concave or funnel-shaped.

In examples a face of the projection may comprise at least one groove in its surface extending from the outlet, or comprises a plurality of grooves in its surface extending from the outlet.

In examples the projection may be substantially circular, and the face may be substantially circular.

In examples embodiments the diaphragm may be retained upon the projection by a ring.

In examples the diaphragm may be deformable into its second configuration under arterial pressure, but not under venous pressure.

In examples the diaphragm may be deformable into its second configuration under venous pressure and alterable into a third configuration under arterial pressure, and when the diaphragm is in its third configuration the indicator is not powered.

In examples, in its third configuration the diaphragm is ruptured.

In examples a male conduit portion configured to mate with a cannulas' female recess is one or more of: at least 5 mm in length, at least 6 mm in length, at least 7 mm in length, and at least 8 mm in length. By providing a male conduit portion that is at least 8 mm in length, empty space that is formed when the conduit portion is mated with an 8 mm deep female recess may be minimized.

In examples, a chamber in which the diaphragm deforms may be in fluid communication with the external atmosphere by means of at least one aperture.

In examples the diaphragm may be crimped.

According to another aspect of the present technology there is provided a kit of parts comprising the device of any preceding embodiment described above and a device tester comprising means to pressurise air in the device to deform the diaphragm.

In examples the device tester may comprise a hollow deformable portion that, in use, is deformable under pressure applied by a user to displace air into the device. In examples the hollow deformable portion may be a bulb.

In some embodiments the device tester may comprise a plunger. In examples the plunger may comprise a shaft configured to be inserted into an aperture of the device forming a portion of a flow pathway to the pressure sensor. In examples the plunger may comprise a stopper configured to restrict further movement of the shaft into the device beyond a predetermined point.

In examples, the device may be releasably secured to a cannula comprising the needle. In examples a needle cover for the needle may comprise the device tester, wherein the device tester delivers air into the device via a needle bore of the needle.

According to another aspect of the present technology there is provided a kit of parts comprising: a device substantially as described herein; a cannula comprising a needle, wherein the device is releasably secured to the cannula; and a needle cover provided over a tip of the needle, wherein the needle cover comprises a device tester comprising means to pressurise air in the device via a needle bore of the needle to activate the indicator.

According to another aspect of the present technology there is provided a kit of parts comprising: a device substantially as described herein; a device tester comprising means to pressurise air in the device to activate the indicator of the device; a sealed package containing the device and device tester, wherein at least a portion of the sealed package is deformable so as to permit operation of the device within the sealed package.

In examples, at least a portion of the sealed package is at least translucent. In examples, at least a portion of the sealed package is transparent.

According to another aspect of the present technology there is provided a kit of parts comprising the device substantially as described herein, and a holder configured to hold the device in use.

In examples the holder may comprise a body having a cavity to hold the device by means of a friction fit.

In examples at least one exterior surface of the holder may comprise at least one grip feature.

In examples, at least a portion of the holder may be made of see-through material. In examples, the holder includes at least one opening between an exterior of the holder and the device.

According to another aspect of the present technology there is provided a method of indicating if a needle is positioned in a human or animal vessel or cavity comprising the step of providing a device made in accordance with any embodiment described herein.

The above and other features will become apparent from the following description and the attached drawings.

### BRIEF DESCRIPTION OF THE DRAWINGS

Further aspects of the present disclosure will become apparent from the following description which is given by way of example only and with reference to the accompanying drawings in which:

FIG. 1A is a perspective view of another example of a device made in accordance with an aspect of the present technology.

FIG. 1B is a top view of the device of FIG. 1A.

FIG. 1C is a cross-sectional side view of the device of FIG. 1A.

FIG. 1D is an exploded perspective view of the device of FIG. 1A.

FIG. 1E is a bottom view of the device of FIG. 1A.

FIG. 2A is a top perspective view of an exemplary diaphragm used in devices made in accordance with an aspect of the present technology.

FIG. 2B is a bottom view of the diaphragm of FIG. 2A.

FIG. 2C is a top view of the diaphragm of FIG. 2A.

FIG. 2D is a side view of the diaphragm of FIG. 2A.

FIG. 2E is a cross-sectional side view of the diaphragm of FIG. 2A.

FIG. 3A is a top perspective view of an exemplary switch component used in devices made in accordance with an aspect of the present technology.

FIG. 3B is a side view of the switch component of FIG. 3A.

FIG. 4 is a diagrammatic view of example of a device made in accordance with an aspect of the present technology.

FIG. 5 is a diagrammatic view of example of a device made in accordance with an aspect of the present technology.

FIG. 6A is a side cross-section view of a holder for a device made in accordance with an aspect of the present technology.

FIG. 6B is a plan cross-section view of the holder of FIG. 6A illustrating how a device is retained within the holder.

FIG. 7A is a cross-section of a first device tester made in accordance with an aspect of the present technology.

FIG. 7B is a cross-section of the first device tester of FIG. 7A illustrating how it attaches to a device made in accordance with an aspect of the present technology.

FIG. 8A is a cross-section of a second device tester made in accordance with an aspect of the present technology.

FIG. 8B is a cross-section of the second device tester of FIG. 8A illustrating how it attaches to a device made in accordance with an aspect of the present technology.

FIG. 9 is a side view of an exemplary needle cover comprising a testing unit in accordance with an aspect of the present technology.

FIG. 10 is a plan view of packaging for an exemplary a testing unit affixed to an exemplary device in accordance with an aspect of the present technology.

FIG. 11A is an exploded view of the components of a device made in accordance with an aspect of the present technology;

FIG. 11B is a perspective view of the device of FIG. 11A when assembled.

FIG. 12 is plan view of a conductive switch component of the device of FIG. 11A.

FIG. 13A is a plan view of the switch component of FIG. 12 when assembled with a battery and a spacer.

FIG. 13B is a side view of the assembly of FIG. 13A.

FIG. 14A is a cross-section of the device of FIG. 11B when attached to a cannula (for clarity an upper portion of the casing of the device is not shown).

FIG. 14B is close-up of the cross-section of the device of FIG. 11B as circled in FIG. 14A.

FIG. 15 is an exploded view of the components of a device made in accordance with a further embodiment of the present technology invention.

FIG. 16A is a cross-section of a device made in accordance with another embodiment of the present technology.

FIG. 16B is a perspective cross-section of a device made according to similar principles as the device of FIG. 16A.

### DETAILED DESCRIPTION

Although in the present embodiment the device is a blood vessel entry indication device the technology is not limited to detecting blood pressure for the purpose of cannulation. In other embodiments a device embodying the technology could be employed for detecting the entry of a needle into any other liquid containing vessel or cavity of the body such as the bladder or spinal cord. Some embodiments of the technology may be intended for use with gaseous fluids.

It should therefore be noted that in this specification the word “fluid” is intended to have its normal meaning of a flowing substance such as a liquid or gas, and is not intended to be limited to a liquid such as blood.

### Operating Parameters

In the use case of arterial and venous cannulations, exemplary devices according to aspects of the present technology are configured such that at least one indicator is activated when pressure acting on the pressure sensor is at or above a minimum threshold. Exemplary devices according to aspects of the present technology have been developed to provide a reliable indication of correct positioning of the needle when considering that training and professional advice directs the use of a tourniquet when performing peripheral venous cannulations.

The torniquet increases Peripheral Venous Pressure (PVP), whilst surrounding tissue pressure remains about the same. PVP ranges from approximately 10 to 25 cmH₂O in a healthy patient, but could be as low as 2 to 3 cmH₂O in a critically unwell patient. With a tourniquet applied, even poorly or on a patient with very low blood pressure, the minimum PVP may be in the order of 25 cmH₂O. Subcutaneous tissue pressure can range from about 1 cmH₂O to about 13.5 cmH₂O: subcutaneous tissue pressure around the vein/artery is up to 4 cmH₂O without a tourniquet, the subcutaneous tissue pressure is up to 6 cmH₂O with a tourniquet, and the subcutaneous tissue pressure is up to about 13.5 cmH₂O in a swollen limb.

As such, the exemplary devices of the present technology have been configured to activate the at least one indicator under all pressures that have a very high likelihood of being indicative of the needle being located in a vein or artery. In examples, the minimum threshold may be about 25 cmH₂O. More preferably, the minimum threshold may be about 30 cmH₂O.

In addition to accuracy with respect to the magnitude of the pressure, the rate of response of the device—i.e., the speed at which the indicator is activated and/or deactivated in response to a change in pressure—is considered to be highly important for clinical utility. The purpose of the device is to provide a user with feedback as to correct placement of the intravascular needle. If the indicator does not activate or deactivate in good time, there is an increased risk that the needle may be incorrectly placed. As such, exemplary devices of the present technology may be configured to activate and/or deactivate the indicator in less than about one (1) second of exceeding or dropping below the minimum threshold pressure value. Generally speaking, the smaller the pressure differential between vein and tissue, the slower the response time. As such, it is anticipated that a minimum threshold of greater than 25 cmH₂O (e.g., about 30 cmH₂O) may be helpful in achieving a desirable response time.

Another requirement for clinical utility is that the indicator remain activated through the pressure range that may be expected from a patient while the needle is located in a vein or artery. If the pressure sensor and/or indicator fails under such expected conditions, then the usefulness of the device diminishes. It will be appreciated that various examples of the device may be configured to operate with different levels of upper operating ranges, depending on the acceptable likelihood of failing to the detect the presence of the needle in a vein or artery of outlier patients. For example, it is estimated that 99% of all patients may fall within the pressure range of about 30 cmH₂O to about 250 cmH₂O. As such, it is anticipated that examples of the device may be configured to activate the indicator at all pressures from the minimum pressure to at least about 250 cmH₂O.

For completeness, it should be appreciated that alternative configurations are contemplated. For example, it is estimated that 90% of all patients may fall within the pressure range of about 30 cmH₂O to about 100 cmH₂O. In examples, the device may be configured to activate the at least one indicator at all pressures from the minimum threshold through to least 100 cmH₂O. As a further example, the device may be configured to configured to activate the at least one indicator from the minimum threshold through to a value above about 250 cmH₂O—for example at least 500 cmH₂O—in order to increase the likelihood of the device functioning for any given individual. This is not intended to exclude embodiments in which the device activates the indicator above this value, but rather emphasise an intention that the device continue to function up to at least that point.

### Needle Entry Indicator Device

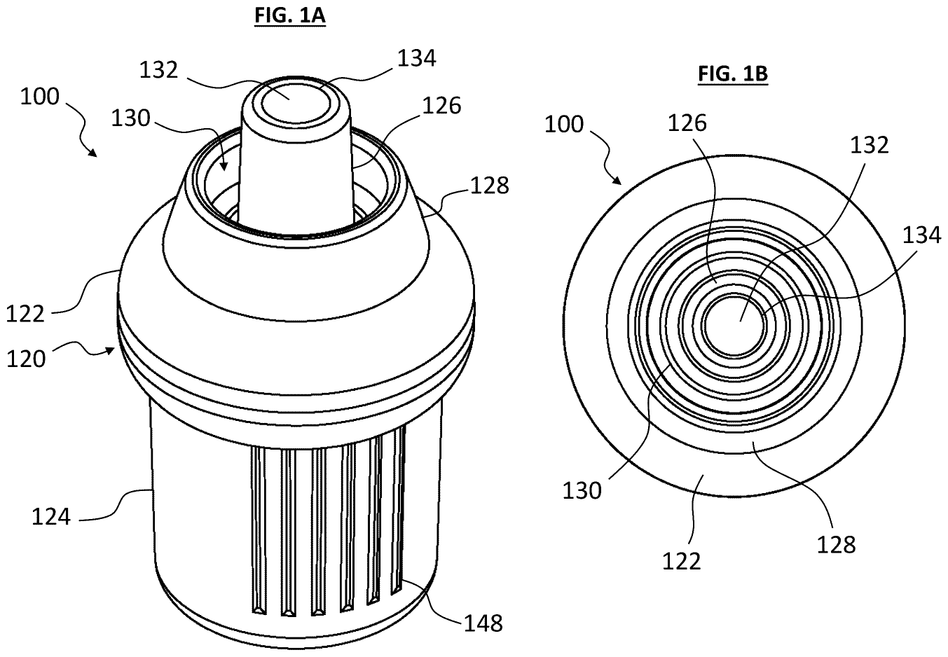

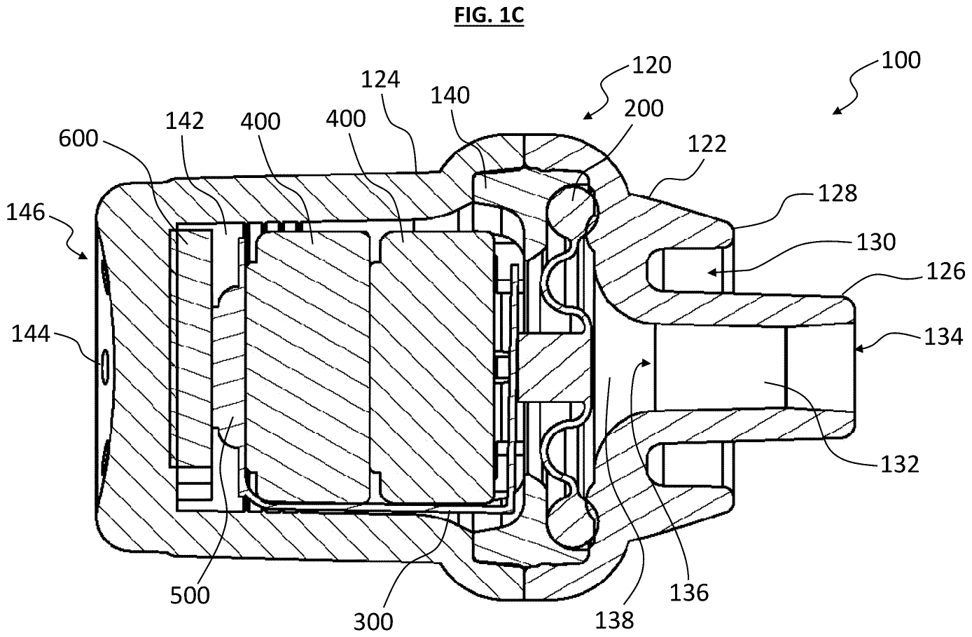

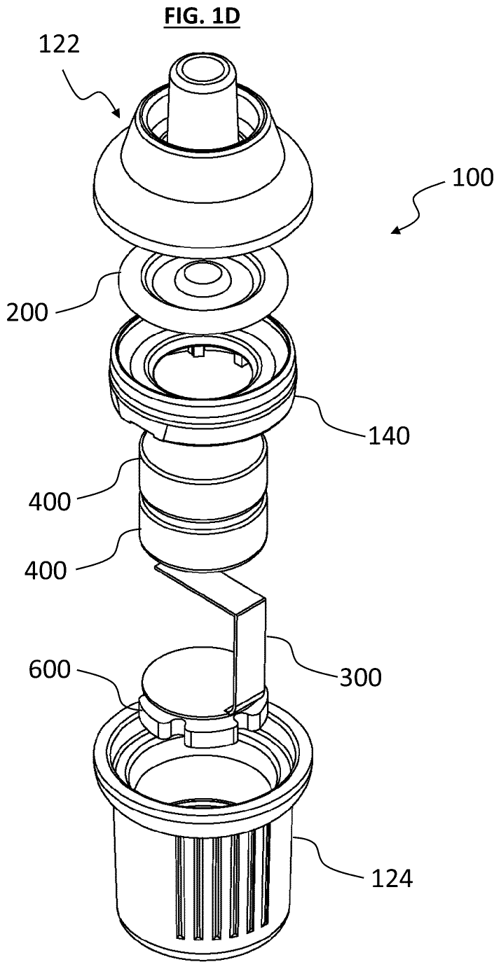

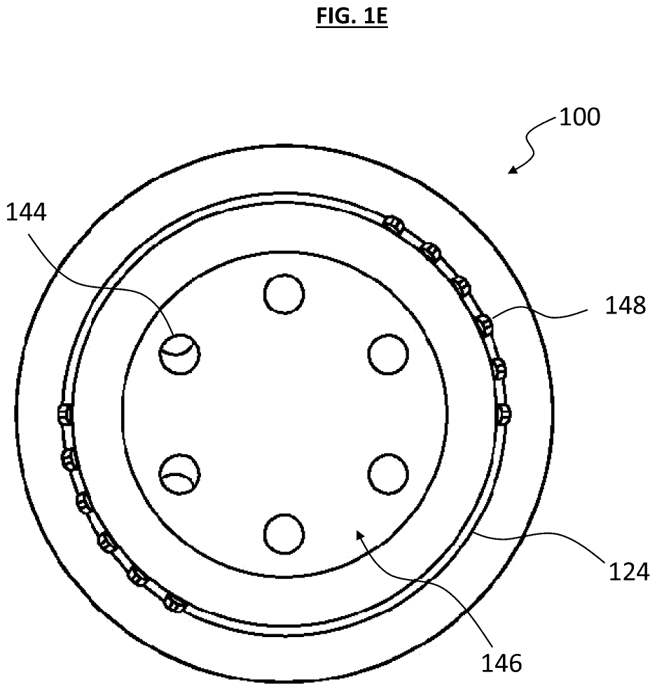

FIGS. 1A to 1E illustrate an exemplary device 100 for indicating entry of a needle into a body cavity, duct, or vessel of a human or non-human animal. In this example the device 100 comprises a body 120, comprising a first body portion 122 and a second body portion 124. The first body portion 122 comprises a male conduit portion 126 surrounded by an annular ridge 128 defining an annular recess 130 for receiving a cannula (not shown) such that a Luer taper connection is formed. A fluid port 132 in the male conduit portion 126 comprises an inlet 134 at an exterior of the body 120 and an outlet 136 at a first internal cavity 138 of the body 120.

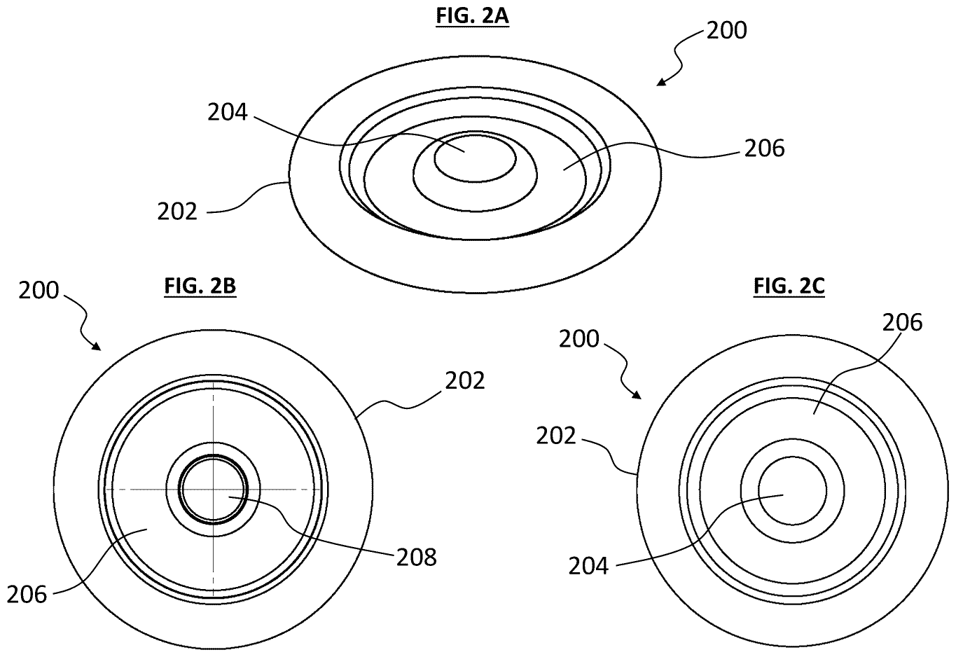

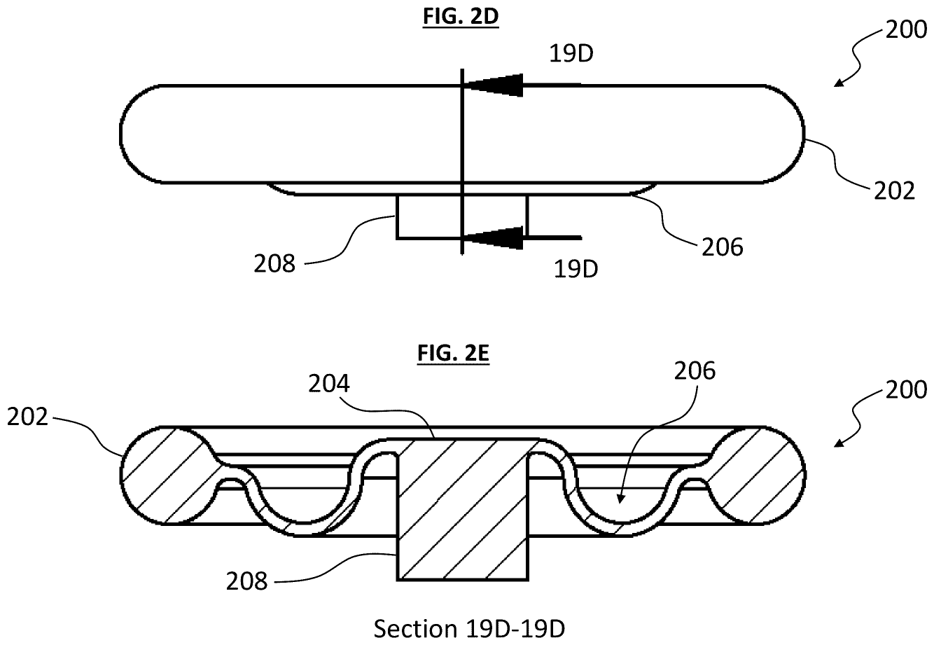

Referring to FIGS. 1C and 1D, an annular diaphragm retention member 140 is held in place internally between the first body portion 122 and the second body portion 124. The diaphragm retention member 140 in turn clamps a diaphragm 200 against the first body portion 122 to retain it in place. Referring to FIGS. 2A to 2E, in this example the diaphragm 200 includes an outer annular rim 202, a central portion 204, and an annular corrugation 206 extending between the annular rim 202 and the central portion 204. A cylindrical protrusion 208 extends from the central portion 204.

Referring to FIG. 1C, the diaphragm 200 is installed such that the protrusion 208 faces away from the fluid port 132. In this example, the inlet 134 and the outlet 136 of the fluid port 132 are axially aligned with the central portion 204 of the diaphragm 200. The surface of the diaphragm 200 from which the protrusion 208 extends faces a second internal cavity 142. A switch unit 300, batteries 400, and spacer 600 are provided in the second internal cavity 142.

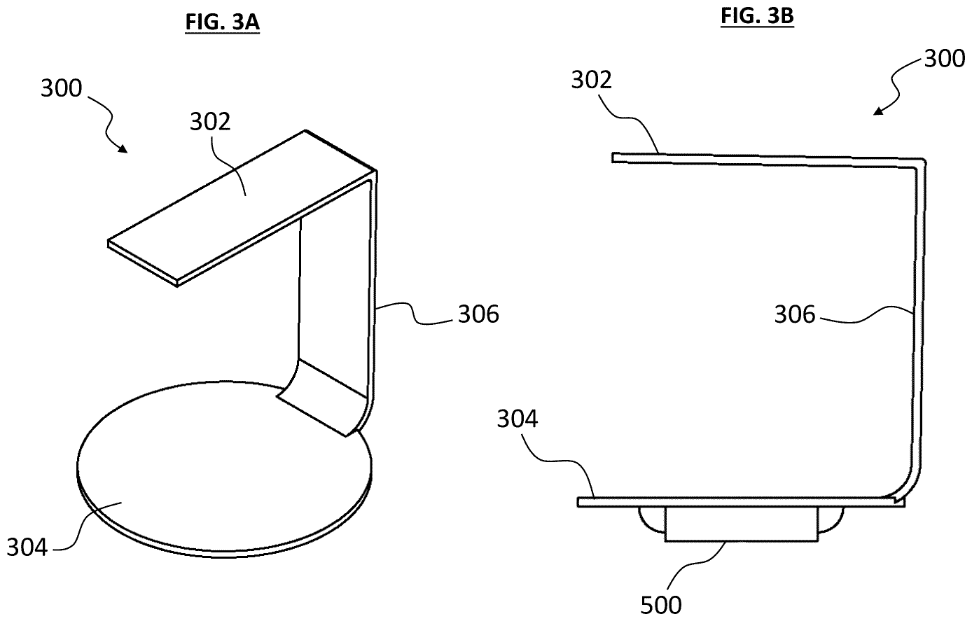

Referring to FIG. 3A and FIG. 3B, the switch unit 300 is made of a flexible circuit board having conductive tracks printed thereon. The switch unit 300 includes a first contact portion 302, a second contact portion 304, and a bridge portion 306 between the first contact portion 302 and the second contact portion 304. An indicator in the form of LED 500 is provided to second contact portion 304. In examples the LED 500 may output light in the visible light spectrum. However, alternative examples are contemplated in which the light is in the non-visible spectrum (e.g., near-infrared or infrared light)—for example, for use in conjunction with a night-vision device.

As shown in FIG. 1C, two batteries 400 are positioned between the first contact portion 302 and the second contact portion 304. In this example the batteries 400 are alkaline in order to facilitate disposal of the device 100, particular in single-use cases. In this example the batteries 400 are stacked in series, enabling a sufficiently high voltage to be achieved while maintaining a narrow diameter footprint. For example, the batteries 400 may be LR41 button cell batteries—although it should be appreciated that this is not intended to be limiting to all embodiments of the present technology.

A spacer 600, in this example made of a silicone material, is provided between the LED 500 and the distal end of the second internal cavity 142, to assist in achieving a desired positioning of the switch unit 300 relative to the diaphragm 200 and the batteries 400.

In use, once pressure from fluid entering the first cavity 138 reaches a minimum threshold, or activation pressure, this pressure displaces diaphragm 200 such that protrusion 208 presses first contact portion 302 of the switch unit 300 into contact with the proximal battery 400 and complete the circuit to turn on LED 500. Because the circuit is only completed to activate LED 500 under targeted pressures, there is no power draw on the batteries 400 while the device 100 is not actively being used or the pressure is below the minimum threshold. This means that the battery life of the device 100 is preserved for a greater period of time than devices which constantly draw power to monitor pressure. This may allow the use of smaller capacity batteries, potentially reducing footprint and/or weight and/or cost of the device as a result, or may allow for a longer shelf-life of the device, or may avoid the need for power saving measures which would otherwise increase complexity and cost of the device.

In the use case of arterial and venous cannulations, the device 100 is configured such that the LED 500 is turned on while pressure within the first cavity 138 (i.e., acting against diaphragm 200) is within the range of about 30 cmH₂O to at least 500 cmH₂O. For completeness, this configuration means that: (1) the LED 500 is turned on once pressure reaches 30 cmH₂O, (2) the LED 500 remains on while the pressure is at least 30 cmH₂O or above, (3) the device is capable of keeping the LED 500 activated while experiencing pressure of 30 cmH₂O up to at least 500 cmH₂O, and (4) the LED 500 is deactivated when the pressure is below 30 cmH₂O.

In examples, the second body portion 124 comprises air ports 144 between the second cavity 142 and an exterior of the body 120. The air ports 144 allow the pressure in the second cavity 142 to equalise with atmosphere, allowing for use of the device 100 at various altitudes and enabling transport at outlier pressures (for example, in aircraft) without damaging the membrane of the diaphragm. In this example the air ports 144 are provided through an end wall 146 of the second body portion distal from the fluid port 132. Positioning of the air ports 144 at this location ensures they are away from the welding site between the first body portion 122 and the second body portion 124.

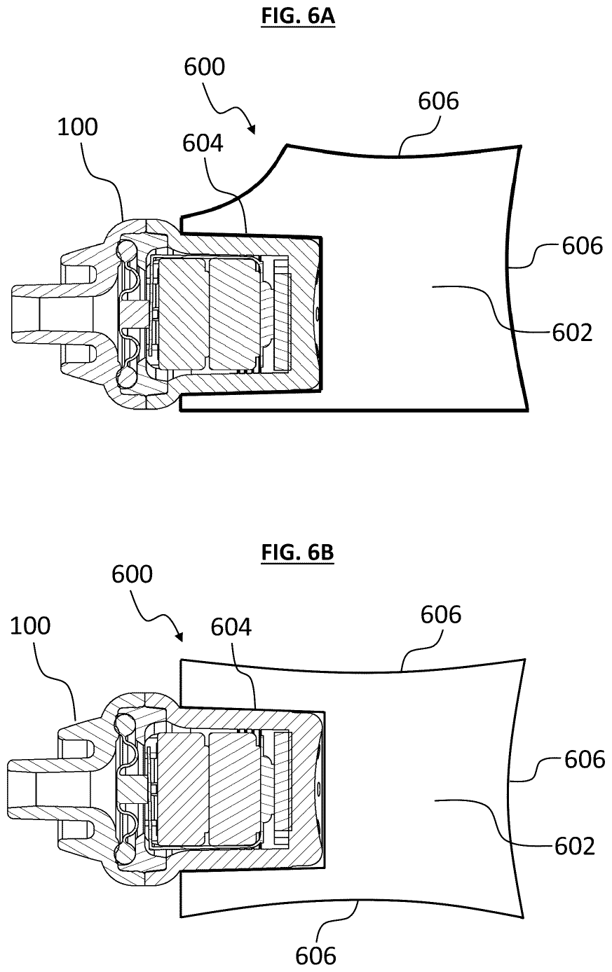

External ridges 148 on the exterior of the second body portion 124 provides grip, whether by a user's fingers or a holder as generally described with reference to FIGS. 6A and 6B.

Devices constructed in the manner described above with respect to FIGS. 1A to 1E were tested to ensure correct operation. A hydraulic test jig was established, comprising a cylindrical reservoir having a dry break valve into which the devices were inserted to expose the diaphragm to pressure within the reservoir. The reservoir was filled to a fill line with simulated blood, and pressurised to a targeted pressure value using a pneumatic pump via a Schrader valve into a headspace. Three devices were tested at 20 cmH₂O and 25 cmH₂O to ensure the device does not activate below the design pressure of 30 cmH₂O. The testing procedure was repeated, with the test pressure increased in increments of 10 cmH₂O from 30 cmH₂O to 250 cmH₂O. After testing at 250 cmH₂O, the test pressure increased in 50 cmH₂O increments until the maximum pressure of 500 cmH₂O was reached. This was repeated three times for each device. This testing procedure was then repeated with three devices.

Initially, the response time was going to be recorded for each test to ensure activation/deactivation times were less than one second. However, it was observed that the LED activation was near instantaneous, and results were primarily dependent on the test operator's reaction time which would give misleading results. The decision was made to instead only record the activation and deactivation using a pass/fail criteria based on the device activating and deactivating immediately in response to the pressure changes.

Overall, all three devices functioned correctly. The LED did not activate at pressures below 30 cmH₂O. The devices all worked consistently from the design pressure of 30 cmH₂O, throughout the range of pressures, up to the maximum pressure of 500 cmH₂O. All three devices also successfully deactivated instantly when the dry break valve was closed.

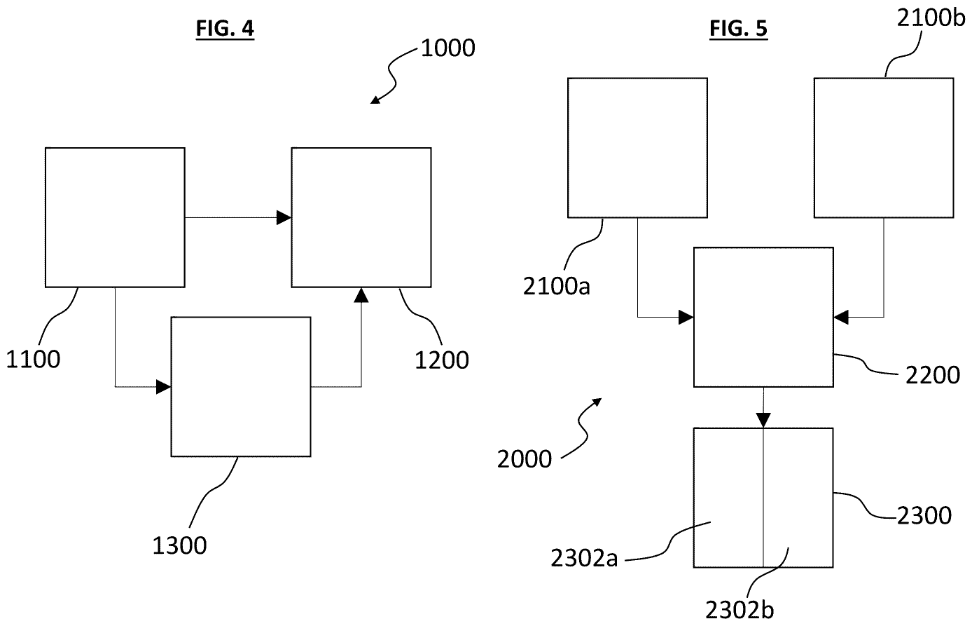

FIG. 4 schematically illustrates another exemplary device 1000 for indicating entry of a needle into a human or animal vessel or cavity. In this example, the device 1000 comprises a digital pressure sensor 1100 in fluid communication with a needle bore of the needle in use. In examples, the digital pressure sensor 1100 may be configured as a pressure switch, outputting a signal to activate an indicator 1200 (e.g., an LED) when the sensed pressure is within a target pressure range—for example between about 30 cmH₂O to about 500 cmH₂O. In an alternative example, the pressure sensor 1100 may output a signal indicative of pressure to a controller 1300, and the controller may selectively activate the indicator 1200 based on a determination of whether the pressure is from about 30 cmH₂O to about 500 cmH₂O.

FIG. 5 schematically illustrates another exemplary device 2000 for indicating entry of a needle into a human or animal vessel or cavity, more particular during central line (or central venous catheter) placement. In central line placement, complications with accidental arterial puncture are widely recognised. In a first embodiment, the device 2000 comprises a first pressure sensor arrangement 2100a in fluid communication with a needle bore of the needle in use, having two diaphragms, each diaphragm configured to be deformable under fluid pressure from a first configuration to a second configuration. The first diaphragm is configured to be deformed to the second configuration when the pressure is within a first pressure range, and the second diaphragm is configured to be deformed to the second configuration when the pressure is within a second pressure range. In examples, the first pressure range may be indicative of central venous pressure (CVP). In examples the second pressure range may be indicative of non-CVP. In examples, the first pressure range may be from about 3 cmH₂O to about 40 cmH₂O. in examples the second pressure range may be from about 40 cmH₂O to about 500 cmH₂O.

The device 2000 includes circuitry 2200 to selectively control activation of an indicator 2300 having first indicator output 2302a and a second indicator output 2302b, depending on actuation of the first and/or second diaphragm. For example, the indicator 2300 may be a multicolour LED capable of selectively outputting green light and red light. In use, the green light is activated when the pressure is within the first range—i.e., indicating that the needle is correctly positioned within the vein. The red light is activated when the pressure increases to the second range—i.e., indicating that an arterial puncture has occurred, to guide remedial action.

In an alternative example, the device 2000 comprises a second pressure sensor arrangement 2100b in fluid communication with a needle bore of the needle in use, in the form of a digital pressure sensor. In examples the pressure sensor 2100b may output a signal indicative of pressure to a circuitry 2200 comprising a controller, and the controller 2200 may selectively activate the first indicator output 2302a or the second indicator output 2302b based on a determination of whether the pressure is within the first pressure range or the second pressure range.

### Device Holder

Turning to FIGS. 6A and 6B, a holder 600 for a device 100 made in accordance with the present technology, holder 600 comprises a body 602 having a cavity 604 that is configured to snugly house a device 100 therein. The cavity 604 is sized so as to retain device 100 by means of a friction fit.

Body 602 is formed of a see-through material so as to allow a user to observe when an LED of device 100 is activated. A see-through material may be transparent or translucent. In some embodiments only part of the body 602 may be see-through, or may include windows or openings to permit viewing of the LED. In other embodiments where a device 100 comprises an audible indicator an opaque material may be used to form a body.

The body 602 is ergonomically designed so as to allow a user to readily handle a device housed therein in difficult conditions, for example on a battlefield. For example, grip features in the form of indents 606 may be provided on the exterior of the holder 606.

### Device Tester

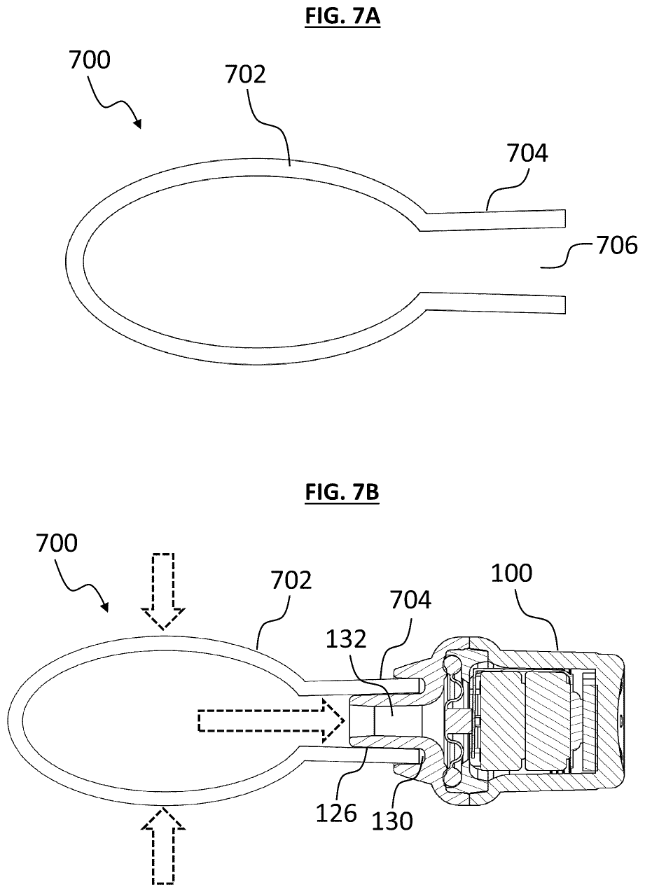

Turning to FIGS. 7A and 7B, a first device tester 700 comprises a hollow bulb 702 having a tubular connector 704 having inner bore 706 that is adapted to snugly house and form an airtight fit with a male conduit portion 126 of a device 100.

Prior to use of device 100 a user may attach device tester 700 to the male conduit portion 126 and squeeze bulb 702 so as to inject air into device 100 and observe if the indicator thereof is powered, and to thereby test whether or not device 100 is operational. Bulb 702 is sized so as to inject sufficient air into device 100 to deform the diaphragm of device 100 and operate its indicator whilst at the same time inject insufficient air to the damage the diaphragm.

In some embodiments the device tester 700 may be attached to a male conduit portion 126 of a device 100 by a breakable connection.

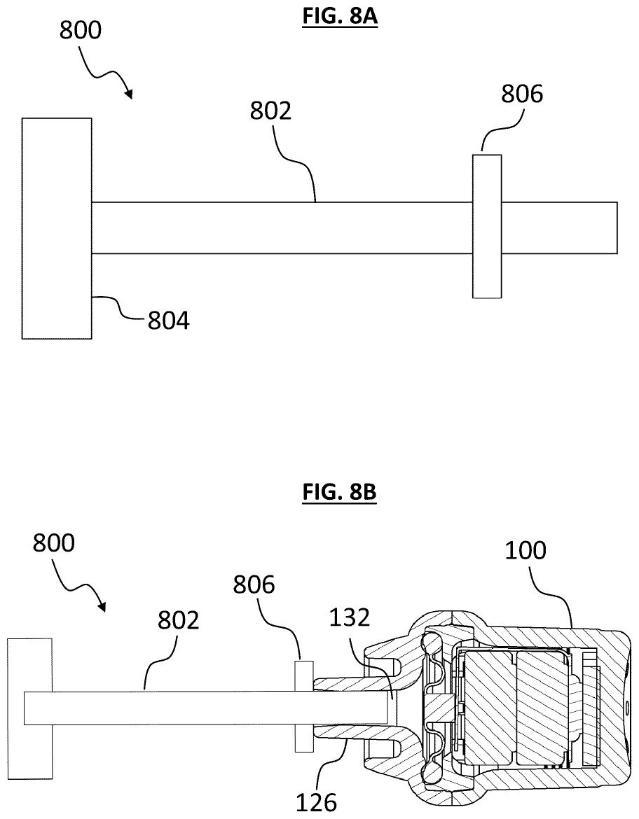

FIGS. 8A and 8B illustrate a second device tester in the form of plunger 800 having a shaft 802, a handle 804 and a stopper 806. Shaft 802 is adapted to be snugly fitted inside a male conduit portion 126 of a device 100 to thereby inject air into device 100 to test the operation of the diaphragm thereof. Stopper 806 is provided on shaft 802 so as to prevent excessive pressure being placed on the diaphragm and consequential damage thereto.

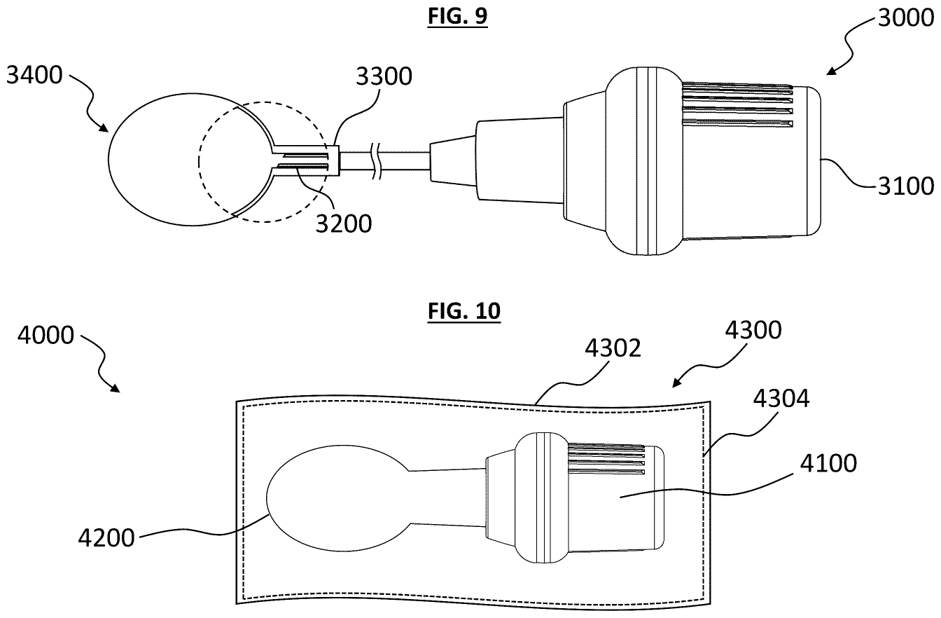

Referring to FIG. 9, in examples an assembly 3000 comprises a device 100 according to the present technology releasably secured to a cannula comprising a needle 3200. In this example a needle cover 3300 for the needle 3200 comprises an integral device tester 3400 in the form of a bulb. The device tester 3400 may be manipulated to deliver air into the device 3100 to test operation before removing the cover 3300 and exposing the needle 3200.

Referring to FIG. 10, a kit of parts 4000 comprises a device 4100, and a device tester 4200 releasably secured to the device 4100 and comprising means to inject air into the device 4100 to test its operation. The combination of the device 4100 and the device tester 4200 are provided within a sealed package 4300. The sealed package 4300 is deformable so as to permit operation of the device tester 4200 within the sealed package 4300, and is at least translucent to enable operation of the indicator to be viewed. For example, the sealed package 4300 may comprise a base portion 4302 and a translucent or transparent window portion 4304. This allows for the device 4100 to be tested prior to opening the sealed package-facilitating ease of disposal should the device 4100 fail testing.

### Further Examples of Needle Entry Indicator Device

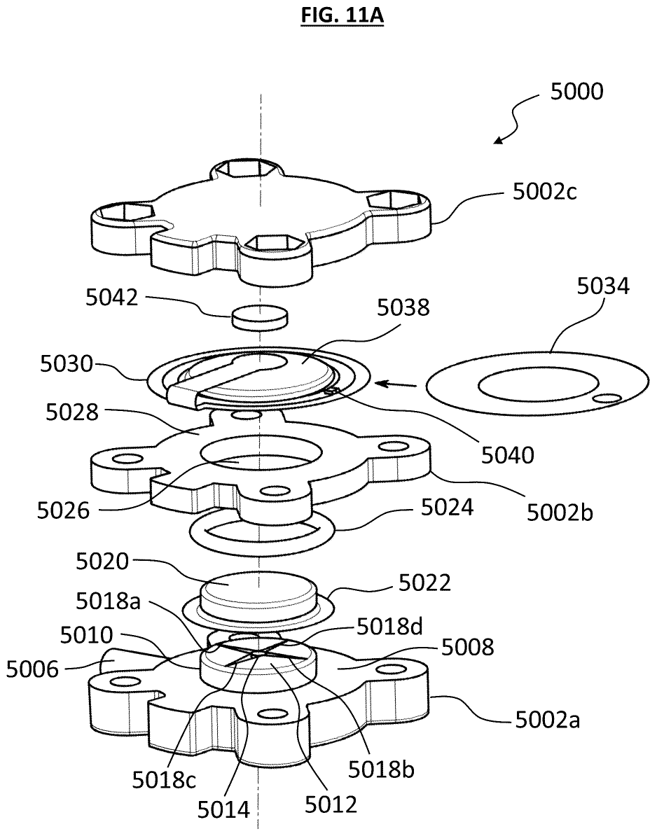

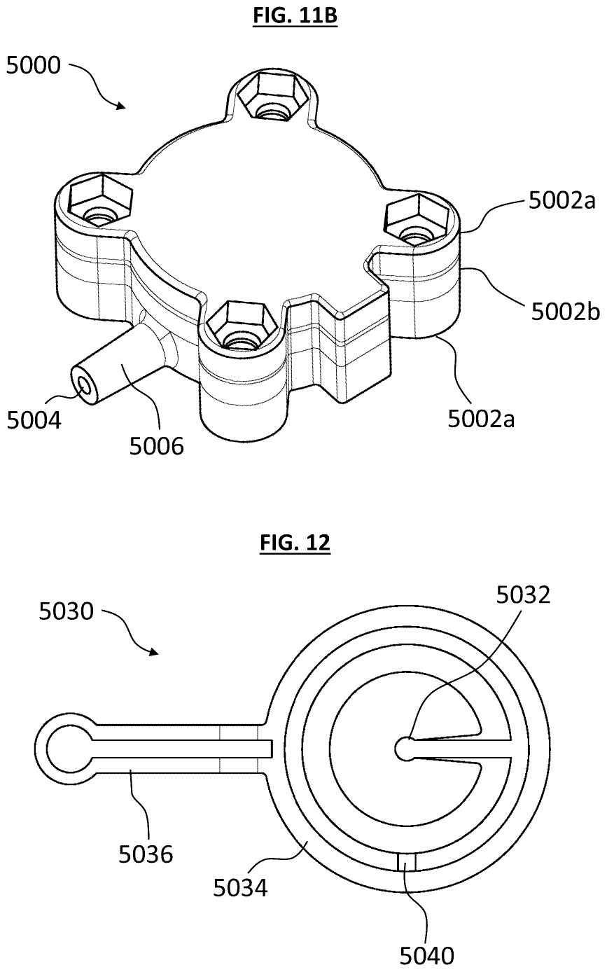

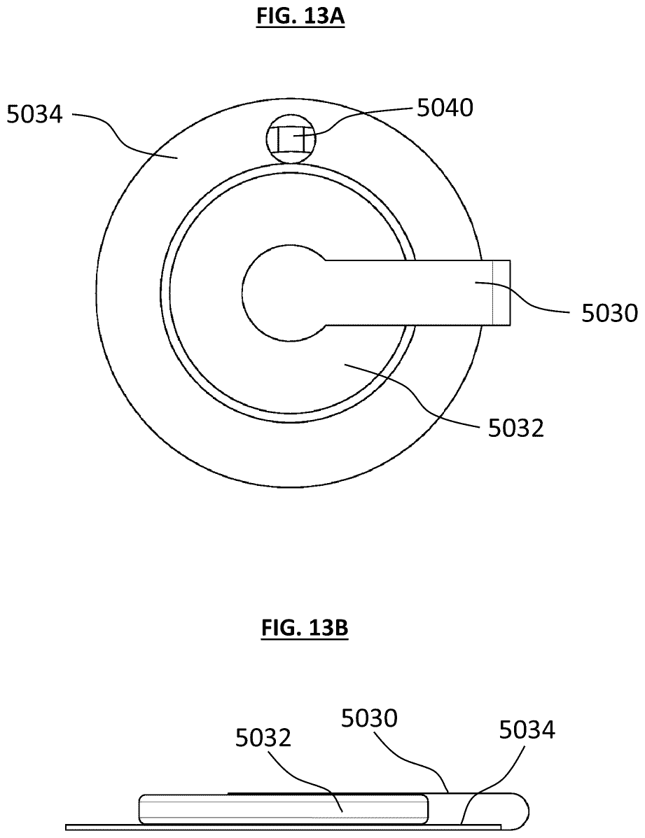

Referring to FIGS. 11A and 11B, a device 5000 made in accordance with an embodiment of the present technology comprises a casing 5002 formed of a lower portion 5002a, a middle portion 5002b and an upper portion 3. Casing 5002 is made of a see-through plastics material.

Lower portion 5002a comprises a fluid inlet 5004 located at a free end of elongate tapered male conduit portion 5006. Conduit portion 5006 extends from a side of lower portion 5002a. On an upper face 5008 of lower portion 5002a is located cylindrical disc-shaped projection 5010, which has a substantially circular upper surface 5012.

Located substantially centrally in upper surface 5012 is fluid outlet 5014, which is fluid communication with fluid inlet 5004 by means of an internal conduit 5016. Formed in upper surface 5012 of cylindrical projection 5010 and extending from fluid outlet 5014 are four grooves 5018a, 5018b, 5018c, 5018d, which each extend radially towards the circular peripheral edge of upper surface 5012.

Diaphragm 5020 is configured to house projection 5010 and further comprises a peripheral flange 5022 that sits on upper face 5008 of lower portion 5002a and extends away from projection 5010 when device 5000 is assembled.

Diaphragm 5020 is positioned over projection 5010 and secured in place by means of rubber ring 5024, which fits around projection 5010 and sits on flange 5022 of diaphragm 5020. Flange 5022 of diaphragm 5020 is thereby sandwiched between rubber ring 5024 and upper face 5008 of lower portion 5002a.

Middle portion 5002b of casing 5002 comprises a substantially disc-shaped chamber 5026 configured to house projection 5010 and diaphragm 5020 such that the circular portion of diaphragm 5020 located over circular face 5012 of projection 5010 is substantially flush with an upper surface 5028 of middle portion 5002b.

Seated upon upper surface 5028 of middle portion 5002b, and adhered thereto, is switch unit 5030, battery 5032, and spacer 5034 assembly. Referring to FIG. 12, switch unit 5030 is a flexible printed circuit comprising a first contact 5032, a conductive ring 5034 and a second contact 5036.

When assembled spacer 5034 sits between conductive ring 5034 and battery 5038 and second contact 5036 of switch unit 5030 is folded over such that it makes contact with an upper surface of battery 5032. Spacer 5034 is adhered to conductive ring 5034.

First contact 5032 is in the plane of conductive ring 5034 and is therefore spaced from a lower surface of battery 5032 by the thickness of spacer 5034. First contact 5032 is resiliently flexible, substantially elongate, and comprises a free end. Located on conductive ring 5034 is LED 5040, which is not covered by spacer 5034 owing to an aperture therein. To maintain second contact 5036 in contact with upper surface of battery 5032 a foam pad 5042 is provided between upper portion 5002c of casing 5002 and second contact 5036. When assembled lower, middle and upper portions 5002a, 5002b, 5002c of casing 5002 are screwed together at the four corners thereof (screws not shown).

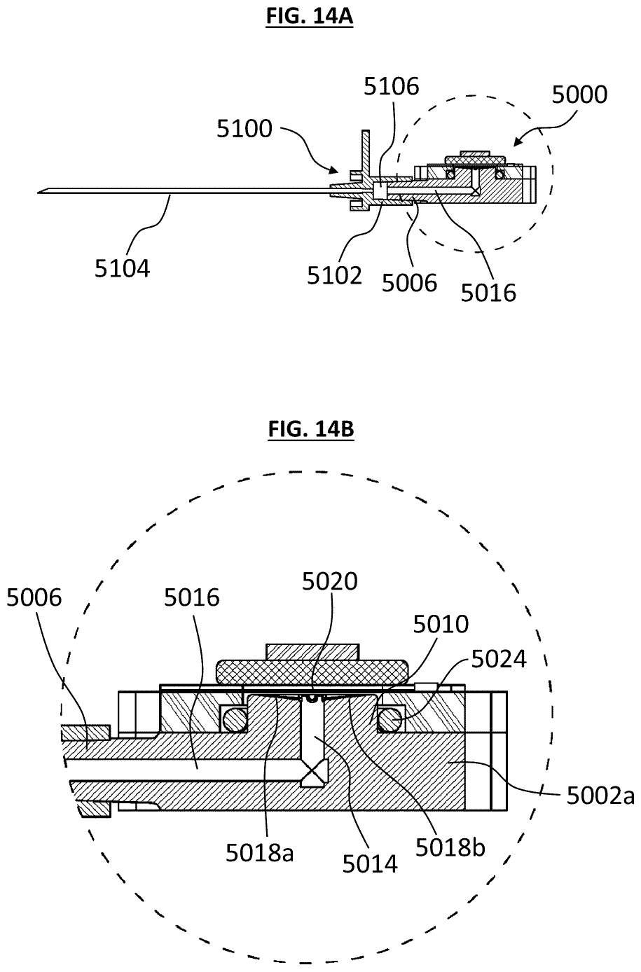

Turning to device 5000 in use, referring to FIG. 14A device 5000 may be attached to a cannula 5100 by insertion of tapered male conduit portion 5006 into a corresponding frustoconical female recess 5102 on cannula 5100 such that a Luer taper connection is formed. Thus needle 5104 is in fluid communication with fluid outlet 5014 via internal conduit 5016.

Needle 5104 is inserted into a patient. When located in a vein or artery blood enters conduit 5016 and exits fluid outlet 5014 deforming diaphragm 5020 deformed from a first (undeformed) configuration to a second configuration wherein it is deformed sufficiently to push first contact 5032 such that it contacts a lower surface of battery 5038 and connects a circuit lighting LED 5040. A user of the device 5000 then knows that the needle 5104 is located in the vein or artery and cannulation may occur.

Should the needle 5104 move out of the vein or artery, the pressure at fluid outlet 5014 drops and diaphragm 5020 returns to its first configuration resulting in the electrical circuit being broken and LED 5014 being deactivated. A user then knows that the needle is not in a suitable position for cannulation to occur.

In use, grooves 5018a, 5018b, 5018c, 5018d inhibit diaphragm 5020 from sticking to upper surface 5012 of cylindrical projection 5010. Beneficially different devices may be adapted to activate at different pressures simply by altering the thickness of spacer 5034. Further, the tension of diaphragm 5020 and thus the pressure at which diaphragm 5020 deforms may be altered by changing the thickness of rubber ring 5024. Furthermore, modifying the thickness and/or length of first contact 5032 of switch unit 5030 may alter the pressure at which LED 5040 is activated.

Referring to FIG. 14A it may be seen that depending upon the dimensions of male conduit portion 5006 and female recess 5102 an empty space 5106 may be formed at the Luer connection. This is undesirable as it results in a delay to activation of LED 5040 as space 5106 is filled.

Thus, it is preferable if male conduit portion 5006 is dimensioned so as to minimize empty space 5106. Female recess is approximately 8 mm in depth and thus to minimize empty space 5106 male conduit portion is at approximately 8 mm in length.

Advantageously a diaphragm may be configured to deform into a second position and power an indicator at a very specific pressure. Thus, in some embodiments a diaphragm may be configured to deform under arterial pressure, but not under venous pressure, thereby allowing a user to determine that a needle is positioned in an artery as opposed to a vein, which can be important when extracting a blood for the purposes of blood gas analysis.

In further embodiments a diaphragm may be deformable into its second configuration under venous pressure and deformable into a third configuration, for example by rupture or distension, under arterial pressure. In the third configuration the indicator would not be powered. Although such an arrangement may irreparably damage a device it would prevent unwanted arterial cannulation.

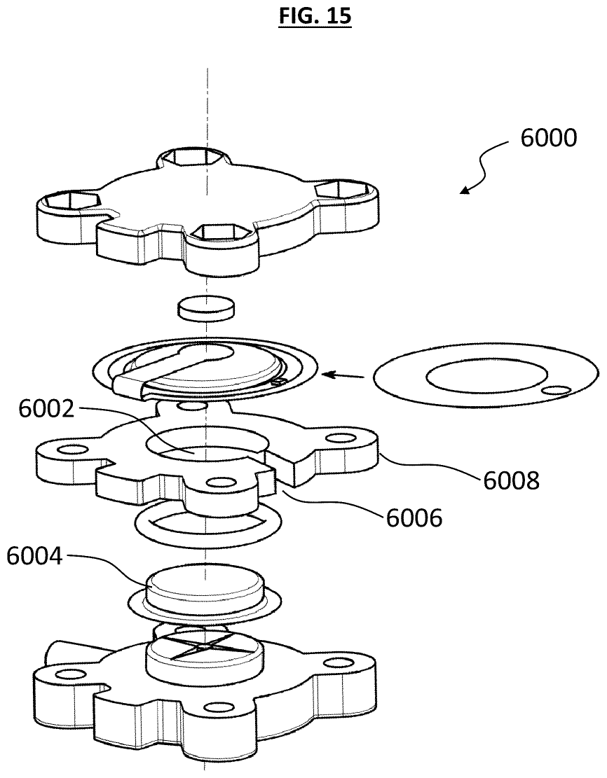

Referring to FIG. 15 and another embodiment of a device made in accordance with the present technology, a device 6000 is largely identical to device 5000, and has a substantially disc-shaped chamber 6002 within which diaphragm 6004 is housed.

Where device 6000 differs from device 5000 is that chamber 6002 is in fluid communication with the outside of device 6000 by virtue of a side wall of middle portion 6008 of its casing being provided with an aperture 6006. Provision of aperture 6006 is preferable to allow diaphragm 6004 freely deform. Aperture 6006 allows further device 6000 to be used at different altitudes with different atmospheric pressures.

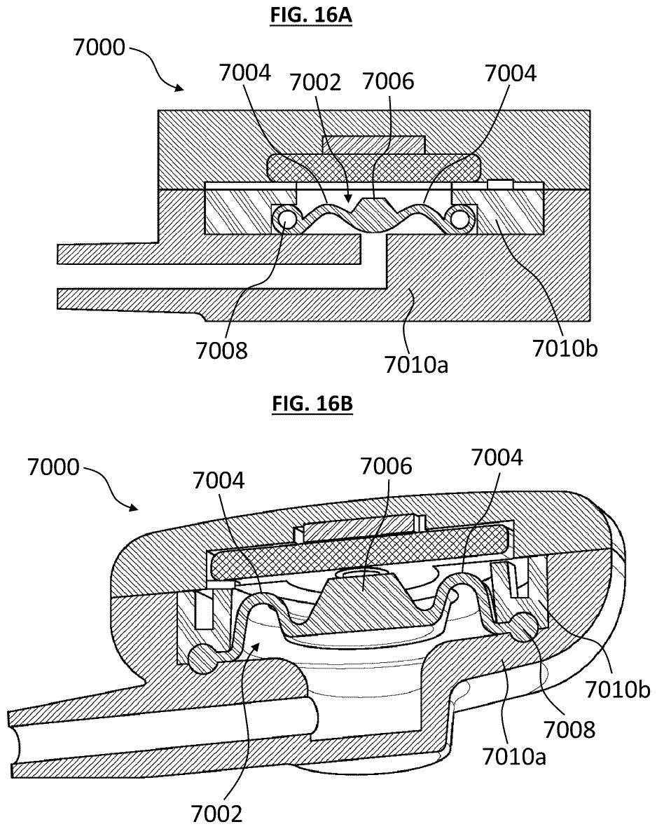

Turning to FIGS. 16A and 16B and a further embodiment of a device made in accordance with the present technology, a device 7000 has a different diaphragm arrangement, with a crimped diaphragm 7002. Diaphragm 7002 is crimped in the sense that it comprises an annular corrugation 7004 that extends in the direction of the switch of device 7000. Diaphragm 7002 further comprises a central raised portion 7006 that extends in the direction of the switch of device 7000 and operates the switch of device 7000 when in device 7000 is use and diaphragm 7002 is deformed by air pressure.

In contrast to the examples of FIG. 11A and FIG. 15, diaphragm 7002 is not positioned over a projection and secured in place by means of rubber ring. Instead, diaphragm 7002 is retained in position over an air inlet by circumferential rib 7008 of diaphragm 7002 being held between a lower portion 7010a of the casing of device 7000 and a middle portion 7010b of the casing of device 7000. Provision of a crimped diaphragm may be beneficial in comparison to a planar diaphragm as a crimped diaphragm is able to move freely, making it more responsive, and is less likely to be hindered from moving by sticking to a surface of the device.

Although in the present embodiment the diaphragms are formed of non-conductive material in other embodiments diaphragms may be formed of an electrically conductive rubber material. For example, a non-conductive diaphragm may be provided with a conductive coating, conductive particles throughout or a conductive connector.

In the present specification the phrase ‘touches a surface’ is intended in its broadest sense to include both at least merely contacting a surface (in the event that the diaphragm is conductive) and also applying pressure thereto (in the event that the diaphragm activates a pressure-operated switch.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the field of endeavour in any country in the world.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The present disclosure may also be said broadly to consist in the parts, elements and features referred to or indicated in the specification of the application, individually or collectively, in any or all combinations of two or more of said parts, elements or features. Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the present disclosure and without diminishing its attendant advantages. It is therefore intended that such changes and modifications be included within the present disclosure as defined by the appended claims.

1. A device to indicate entry of a needle into a body cavity, duct, or vessel of a human or non-human animal, the device comprising:
a pressure sensor configured to be in fluid communication with a needle bore of the needle in use; and
at least one selectively activated indicator,
wherein the device is configured to activate the indicator while pressure acting on the pressure sensor is at or above a minimum pressure threshold, wherein the minimum pressure threshold is about 25 cmH₂O.

2. The device of claim 1, wherein the minimum pressure threshold is about 30 cmH₂O and above.

3. The device of claim 1, wherein the device is configured to activate the at least one indicator at all pressures from the minimum threshold through to at least 100 cmH₂O.

4. The device of claim 1, wherein the device is configured to activate the at least one indicator at all pressures from the minimum threshold through to at least 250 cmH₂O.

5. The device of claim 1, wherein the device is configured to activate the at least one indicator at all pressures from the minimum threshold through to at least 500 cmH₂O.

6. The device of claim 1, wherein the pressure sensor comprises at least one diaphragm that is deformable under fluid pressure from a first configuration to a second configuration, wherein in the first configuration the indicator is not activated, and in the first configuration the indicator is activated.

7. The device of claim 6, wherein in the second configuration the diaphragm closes a switch to activate the indicator.

8. The device of claim 1, wherein the device comprises a body containing the pressure sensor, wherein the body comprises a first internal cavity between a first face of the diaphragm and a fluid port in fluid communication with the needle bore of the needle in use.

9. The device of claim 8, wherein the body comprises a second internal cavity formed in part by a second face of the diaphragm opposing the first face, and at least one air port between the second internal cavity and an exterior of the body.

10. The device of claim 9, wherein the at least one air port is provided in an end surface of the body distal from the fluid port.

11. The device of claim 9, wherein the body comprises a plurality of air ports.

12. The device of claim 8, wherein the fluid port comprises an inlet at an exterior of the body and an outlet at the first internal cavity, and the inlet and the outlet of the fluid port are axially aligned with a central portion of the diaphragm.

13. The device of claim 1, comprising a plurality of alkaline batteries stacked in series along a longitudinal axis between a first end of the body and a second end of the body.

14. The device of claim 1, wherein the pressure sensor comprises a digital pressure sensor.

15. The device of claim 14, wherein the digital pressure sensor is configured as a pressure switch, outputting a signal to activate the indicator when the sensed pressure is above the minimum pressure threshold.

16. The device of claim 15, wherein the digital pressure sensor is configured to output a signal indicative of pressure to a controller, and the controller is configured to selectively activate the indicator based on a determination of whether the pressure is above the minimum pressure threshold.

17. The device of claim 1, wherein the indicator comprises a light configured to output light in the non-visible spectrum.

18. The device of claim 17, wherein the light is configured to output near-infrared or infrared light when activated.

19. A kit of parts comprising:
a device to indicate entry of a needle into a body cavity, duct, or vessel of a human or non-human animal, the device comprising:
a pressure sensor configured to be in fluid communication with a needle bore of the needle in use; and
at least one selectively activated indicator,
wherein the device is configured to activate the indicator while pressure acting on the pressure sensor is at or above a minimum pressure threshold, wherein the minimum pressure threshold is about 25 cmH2O;
a cannula comprising a needle, wherein the device is releasably secured to the cannula; and
a needle cover provided over the needle, wherein the needle cover comprises a device tester comprising means to pressurise air in the device via a needle bore of the needle to activate the indicator.

20. A kit of parts comprising:
a device to indicate entry of a needle into a body cavity, duct, or vessel of a human or non-human animal, the device comprising:
a pressure sensor configured to be in fluid communication with a needle bore of the needle in use; and
at least one selectively activated indicator,
wherein the device is configured to activate the indicator while pressure acting on the pressure sensor is at or above a minimum pressure threshold, wherein the minimum pressure threshold is about 25 cmH2O;
a device tester comprising means to pressurise air in the device to activate the indicator of the device;
a sealed package containing the device and device tester, wherein at least a portion of the sealed package is deformable so as to permit operation of the device tester within the sealed package.
